(19) United States
(12) Patent Application Publication
(10) Pub. No.: US 2007/0282364 A1
(43) Pub. Date: Dec. 6, 2007

(54) METHOD AND SYSTEM FOR HARVESTING DONOR STRIPS

(76) Inventor: ROBERT S. HABER, Shaker Heights, OH (US)

Correspondence Address: DRIGGS, HOGG & FRY CO. L.P.A., 38500 CHARDON ROAD, DEPT. DLBH, WILLOUGBY HILLS, OH, 44094, US

(21) Appl. No.: 11/421,169

(22) Filed: May 31, 2006

(51) Int. Cl. A61B 17/00

(52) U.S. Cl. 606/190

(57) ABSTRACT

A tool and method are provided, wherein prongs with sharpened points extend downward from lower workpiece arms, the sharpened prong points aligned into a linear spaced edge array vertically and generally along a dissection plane. Prong outer curvilinear surfaces engage and move engaged tissue outward away from the dissection plane as workpiece arms are compelled outward and away from each other. In one aspect, tapered prongs move downward vertically through a superficial tissue incision, progressively dissecting the tissue along the dissection plane; and movement of prongs horizontally outward separates the tissue into vertical wall tissue portions, separating follicular unit elements into tissue portions without transection. In another aspect, tissue located at gaps between adjacent spaced prongs is compelled to separate into the first and second vertical wall tissue portions without follicular unit element transection.

### BACKGROUND OF THE INVENTION

This invention relates generally to the field of surgical procedures and articles, and more specifically to a method, system and apparatus for harvesting of dermal donor strips.

It is common among adult males to experience male pattern baldness, wherein upper regions of the male scalp experience the gradual replacement of robust thick and visible hair follicles with miniaturized hair follicles which produce fine hair having significantly smaller diameters and lengths, and thereby producing an aesthetically displeasing bare-scalp appearance. A variety of conventional surgical and other medical techniques and methods are known for reintroducing robust hair follicles into the balding scalp regions, including hair transplantation, wherein robust hair follicles are surgically removed from one skin region (typically from the back and/or side of the head) and relocated to a balding scalp region.

One conventional technique for hair transplantation is dermal strip transplantation, wherein strips of donor skin comprising pluralities of individual robust human hair follicles are surgically removed and incorporated into a balding scalp area, with the intention that the robust hair follicles continue to produce robust hair and otherwise thrive in the balding scalp area after the procedure. However, obtaining donor strips through conventional surgical techniques and methods without causing significant transection of healthy hair follicles and associated dermal tissue is difficult.

More particularly, referring now to FIG. 1, human hair may be described as growing in follicular groups or units 100, each unit 100 comprising one or more terminal (full thickness or robust) hair follicles 102 that project visible hairs 101, sebaceous (oil) glands 104, small arrector pilli muscles 106, nerves 108 and blood vessels 110, all surrounded by a gelatinous band of collagen (the perifolliculum) 112 disposed below an outer epidermis skin layer 114. In order for transplanted terminal hair follicles to thrive and continue to produce robust hair, it is generally desirable that these other elements also be successfully removed from the donor region and transplanted into the balding scalp region along with the individual terminal hair follicles. However, conventional surgical techniques using knives or other bladed instruments typically cause damage to portions of the follicular units located along the donor strip borders, through deep incisions that cut through both the outer epidermis layer 114 and the collagen band 112, thereby injuring, transecting or destroying one or more of the hair follicles 102 and/or other follicular unit elements (in effect, the sebaceous glands 104, arrector pilli muscles 106, nerves 108 and blood vessels 110).

The Sandoval “score and spread” technique is one conventional technique representing an improvement over incising the follicular unit 100 with a deep bladed instrument. The Sandoval technique uses a hemostat to bluntly penetrate the protective and resilient outer epidermis 114 and project through the dermal/epidermal junction 120 into the collagen band 112, wherein subsequent application of firm outward pressure by the tips of the hemostat then forces tissue separation in the collagen band 112, thereby causing separation and distribution of hair follicles 102, sebaceous glands 104, arrector pilli muscles 106, nerves 108 and blood vessels 110 into either a donor strip or remaining non-donor strip tissue.

However, the Sandoval technique has disadvantages. In a first instance, the technique requires the generation of the firm outward pressure forces through outward spreading of the hemostat by a surgeon's fingers, which is an ergonomically difficult and inefficient procedure in most surgical applications; secondly, this technique often results in high forces being applied to small areas of tissue engaged by the tips of the hemostat, resulting in crushing-type injuries to impacted tissues.

What is needed is a new system and method for tissue dissection that provides for ergonomic tissue dissection techniques while minimizing tissue damage.

### BRIEF SUMMARY OF THE INVENTION

In one aspect, a tool is provided, comprising first and second handle elements having upper handles and lower workpiece arms, and a pivot pin rotatably connected to the first and second handle elements between the upper handle and the lower workpiece arm. At least one prong with a sharpened point extends downward from each lower workpiece arm, wherein the sharpened prong points are configured to align into a linear spaced edge array when the workpiece arms are brought together in a closed position, the prongs further aligned vertically over the linear spaced edge array and generally along a center dissection plane. The prongs also have outer curvilinear surfaces configured to engage workpiece tissue and move engaged tissue outward away from the center dissection plane as the first lower workpiece arm is compelled away from the second lower workpiece arm.

In one aspect, each of the prongs taper from a widest top region to the sharpened point. In another aspect, one or both of the at least one prongs are pluralities of spaced apart prongs. In one aspect, bottom portions of the prong pluralities have outer curvilinear surfaces configured to align in prong outer surface planar arrays generally parallel to and spaced from each other and the dissection plane when the workpiece arms are moved away from each other into an open position.

In one example, the prongs each have a downward vertical projection length of from about 6 mm to about 8 mm, a widest top region width dimension of about 2 mm, a widest top region thickness dimension of about 2 mm, and the vertically aligned pluralities of prongs define a dissection strip length of about 1.6 cm.

In one example, the tool further comprises a mechanical power means connected to the upper handles configured to pivot the first upper handle relative to the second upper handle about the pivot pin. In another example, the upper handles are configured for engagement by a user's hand; in one aspect, a resilient spring means connected to the handle elements is configured to urge the first upper handle element to pivot about the pivot pin connection relative to the second upper handle element, the first upper handle thereby urged to pivot away from the second upper handle and the first lower workpiece arm to pivot toward the second lower workpiece arm.

In another aspect, the prongs comprise generally planar lateral edge surfaces aligned vertically with respect to the dissection plane.

In another aspect, a tool for tissue incision is provided, comprising a pair of spaced parallel blades projecting downward from an upper handle element and a depth controller means disposed adjacent to the spaced parallel blades and configured to limit a depth of tissue incision of the spaced parallel blades to from about 1.5 mm to about 2 mm.

In another aspect, a method for tissue dissection is provided, comprising the steps of making a superficial incision into a tissue; aligning first and second pluralities of sharpened prongs vertically generally in a dissection plane above the superficial incision, the prongs having wide upper regions tapering to bottom sharpened points, wherein the sharpened points form a spaced linear puncture point edge; engaging the scored incision with the spaced linear puncture point edge; moving the tapered prongs downward vertically through the superficial incision into the tissue, prong outer surfaces and the sharpened points thereby progressively and gradually dissecting the tissue along the dissection plane with increasing depth of insertion; and moving the first prong plurality horizontally outward relative to the second prong plurality, thereby separating the tissue into a first vertical wall tissue portion engaged by first prong plurality outer surfaces and a second vertical wall tissue portion engaged by second prong plurality outer surfaces, wherein the first vertical wall tissue portion is aligned generally parallel to and spaced from the dissection plane and the second vertical wall tissue portion is aligned generally parallel to and spaced from the dissection plane and the first vertical wall tissue portion.

In another aspect, a method is provided wherein the tissue is skin tissue comprising an upper skin layer and a lower collagen band comprising follicular unit elements, and making a superficial incision into the tissue comprises cutting thorough the upper skin layer and into an upper area of the lower collagen band above the follicular unit elements. In one aspect, the superficial incision is limited to an incision depth of from about 1.5 mm to about 2 mm. In one aspect, the superficial incision is made with a double bladed instrument with a depth controller means.

In one aspect, a method is provided separating the tissue into first and second vertical wall tissue portions by compelling first and second pluralities of follicular unit elements to separate into the first and second vertical wall tissue portions without transection. In another aspect, tissue located at gaps between adjacent spaced prongs is compelled to separate into the first and second vertical wall tissue portions without follicular unit element transection.

In another aspect, a method is provided, extending the first and second pluralities of prongs downward from first and second handle element lower workpiece arms, the handle element having upper handles; rotatably connecting a pivot pin to the handle elements between their upper handle and the lower workpiece arms; and the first prong plurality is moved horizontally outward relative to the second prong plurality by compelling the upper first and second handles toward each other, the workpiece arms pivoting away from each other about the pivot pin in response to compelling the upper handles toward each other.

In one aspect, a method is provided configuring the first and second upper handles for engagement by a user's hand and locating the pivot pin more proximate to the first and second prong pluralities than the first and second upper handles. Compelling the upper handle elements toward each other is accomplished by engaging the first upper handle with a thumb; engaging the second upper handle with fingers opposing the thumb; and squeezing the thumb and opposing fingers together with a squeezing force; the pivot pin leveraging the squeezing force into an outward prong spreading force expended by the prong outer surfaces against the tissue greater than the squeezing force.

### BRIEF DESCRIPTION OF THE DRAWINGS

The drawings constitute a part of this specification and include exemplary embodiments to the invention, which may be embodied in various forms. It is to be understood that in some instances various aspects of the invention may be shown exaggerated or enlarged to facilitate an understanding of the invention.

FIG. 1 is a side perspective illustration of a portion of human dermal tissue.

FIG. 2 is a top plan view of a tool according to the present invention.

FIG. 3(a) is a detail view of a portion of the tool of FIG. 2 as indicated in FIG. 2.

FIG. 3(b) is another detail view of the portion of the tool of FIG. 2 as indicated in FIG. 2, with lower portion workpiece arms shown in a fully-open position relative to each other.

FIG. 3(c) is a side detail view of the portion of the tool of FIG. 2 as indicated in FIG. 2.

FIG. 4 is a flowchart illustration of an exemplary method according to present invention.

FIG. 5 is a side view of a donor strip obtained according to the present invention.

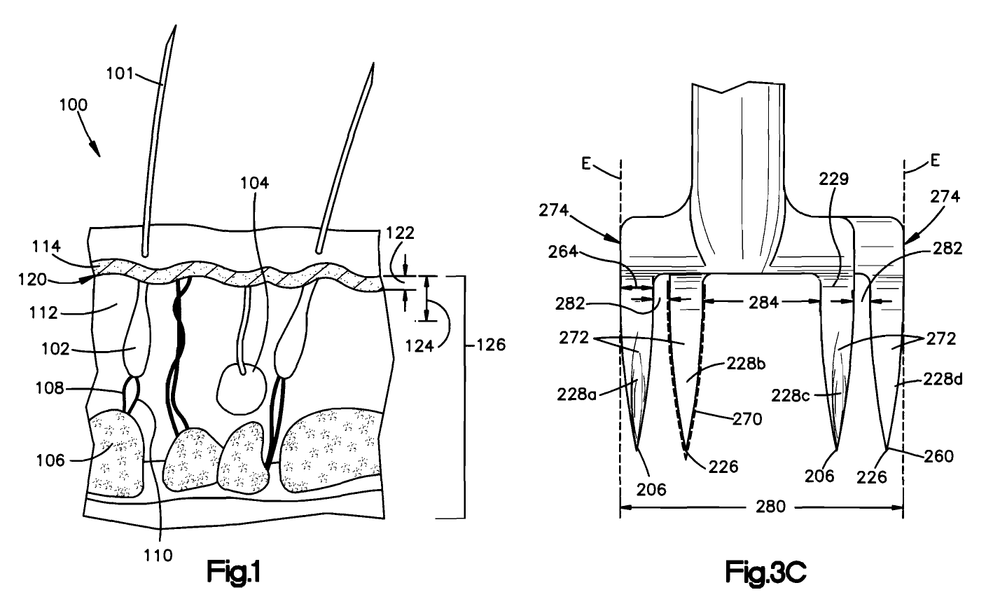

### DETAILED DESCRIPTION OF THE PREFERRED EMBODIMENTS

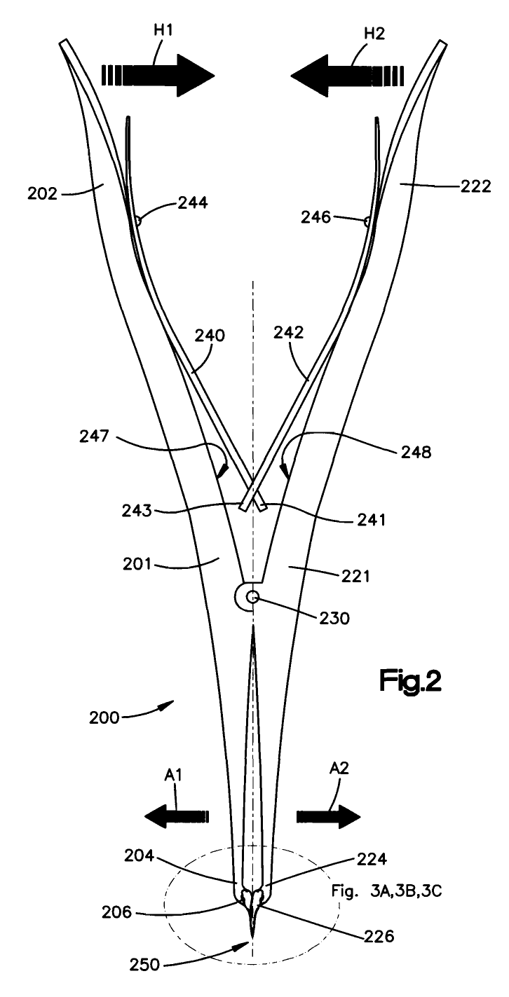

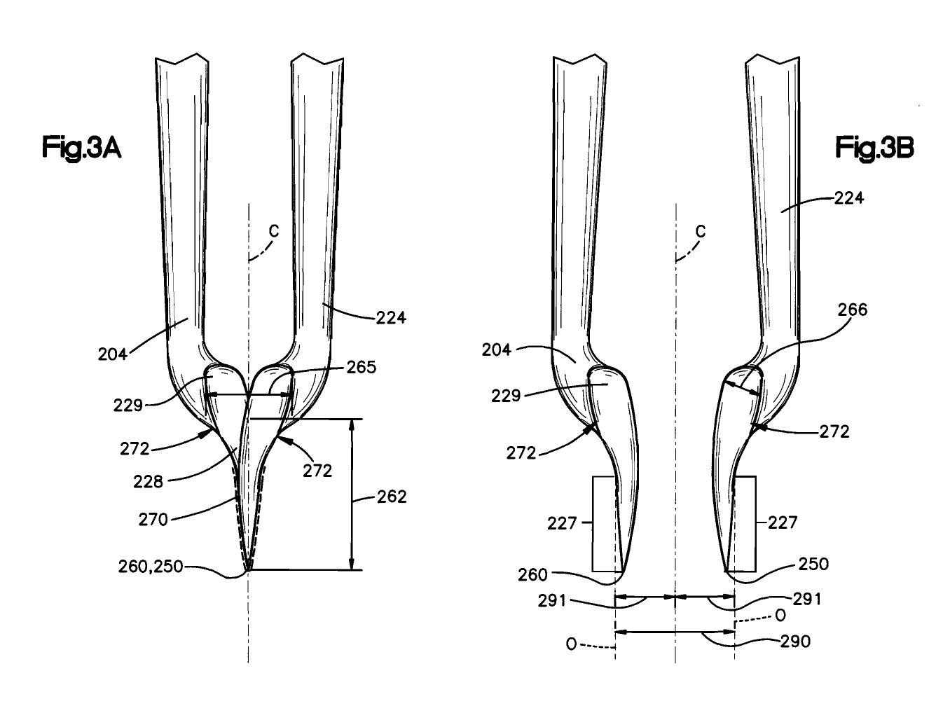

Referring now to FIGS. 2 and 3A through 3C, in one aspect a tool 200 is provided. FIG. 2 provides a top plan view of the tool 200. FIG. 3A provides a detail view of lower workpiece arms 204, 224 according to the top view of FIG. 2. FIG. 3B provides a detail view of the lower workpiece arms 204, 224 taken along the same top view as that provided in FIGS. 2 and 3A, but with the lower workpiece arms 204, 224 shown in a fully-open position relative to each other. And FIG. 3C provides a side view of the lower workpiece arms 204, 224 in a closed position as illustrated in FIGS. 2 and 3A.

A first handle element 201 is pivotably and rotatably connected through a pivot pin 230 to a second handle element 221. The first and second handle elements 201, 221 each have upper handle elements 202, 222 configured for engagement by the thumb and opposing fingers of a user's hand, respectively, and lower workpiece arms 204, 224 arrayed with respect to the pivot point 230, whereby compelling the upper handle elements 202, 222 toward each other (for example, by squeezing the fingers on one of the first and second handle elements 201, 221 toward the thumb engaging the other of the first and second handle elements 201, 221) along directional vectors H1 and H2 results in the workpiece arms 204, 224 being correspondingly urged outward along directional vectors A1 and A2.

In another aspect, the tool preferably incorporates resilient spring members 240, 242 connected to the upper handles 202, 222 through attachment means 244, 246. As will be readily apparent among those skilled in the arts of toolmaking, the bottom regions 241, 243 of the spring members 240, 242 engage each other and/or inner surfaces 247, 248 of the opposing upper handle element 202, 222 as the handle elements 202, 222 are brought together, causing the spring elements 240, 242 to be progressively compelled to bend and deflect toward their respective upper handle elements 202,222. The resilient spring elements 240, 242 will resist this movement with progressively increasing spring forces and, accordingly, when a user relaxes his fingers and opens his grip, the resilient spring elements 240, 242 will urge the upper handle elements 202, 222 outward and back toward the position shown in FIG. 2, wherein the upper handle elements 202, 222 are illustrated in a fully outward position and the lower workpiece arms 204, 224 are brought together in a fully closed position.

At the bottom of the workpiece arms 204, 224 extends a pair each 206, 226 of sharpened staggered prongs 228. Each prong 228 tapers from a widest top region 229 to a sharpened point 260. In one aspect, the prong points 260 are preferably configured to align into and define a common linear point edge 250 when the lower workpiece arms 204, 224 are in the fully closed position. The prong points 260 are also preferably configured to align vertically over the common linear point edge 250 and generally along a dissection plane C passing through the linear point edge 250, wherein prong outer surfaces 272 are aligned into a curvilinear shape 270 that progressively widens from the common linear point edge 250 to maximum width and thickness dimensions 265 at the top of the prongs 228.

In one example, the prong projection length 262 is about 6 mm to about 8 mm, wherein each prong has maximum width and thickness dimensions 264, 266 of about 2 mm each at the top of each prong 228 where it projects from the ends of the lower workpiece arms 204, 224, each prong 228 then progressively tapering in both width and thickness dimensions to form the sharpened points 260.

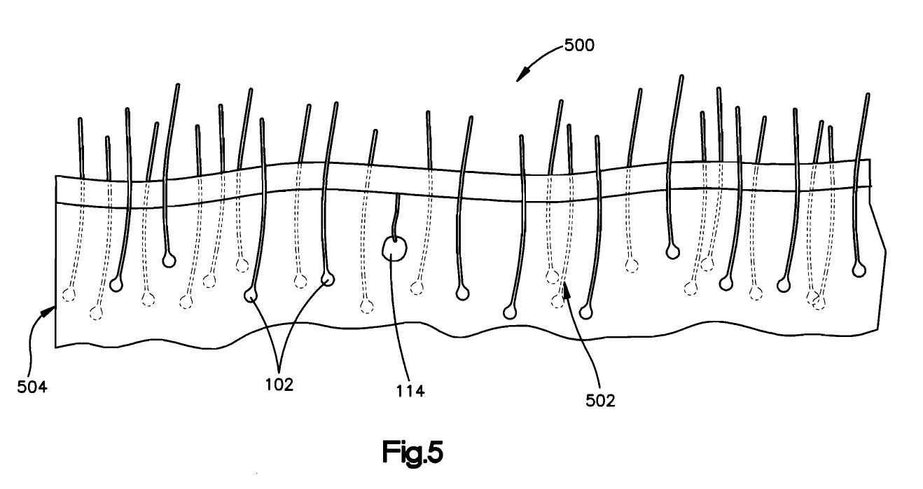

In one aspect, the curvilinear shape 270 is configured to concentrate and distribute downward force from the tool 200 through the leading four prong points 260 along the narrow linear puncture point edge 250 to facilitate puncturing and penetrating the dermal tissues 112, 114, and then progressively separate the tissue 112, 114 with respect to dissection plane C as the tool 200 is compelled progressively downward into the tissue 112, 114, the dissection plane C configured to define a natural dissection plane through the collagen band 112 membrane and thereby provide a clean vertical strip edge wall 502, as illustrated in FIG. 5. In one aspect, downward insertion of the prongs 228 into donor tissue thus progressively and gradually separates the tissues 112 and 114 with increasing depth of insertion, causing less blunt tool impact damage to the engaged tissue 112, 114 compared with conventional techniques that use blunt-tipped instruments such as hemostats.

More particularly, collagen band 112 tissue may easily and naturally separate along a natural dissection plane generally normal to a plane defined by the upper surface skin 114, and thereby provide clean vertical strip edge walls 502 appropriate for transplantation techniques. Thus, the prongs 228 easily separate the gelatinous collagen band 112 membrane and compel the robust hair follicles 102 and other follicular unit elements, such as the sebaceous glands 104, to move into either the donor strip tissue or the remaining tissue without transection. The present method contrasts with conventional techniques using deep incisions with sharpened blades, wherein the sharpened blades will typically transect anything in their path of incision. Separation of collagen band 112 tissues by the prongs 228 thus greatly diminishes the amount of follicular unit element transections during separation of the gelatinous band 112 into donor strip and remaining tissue bodies.

It is also preferred that the prong lateral edge surfaces 274 are generally planar and aligned vertically with respect to the dissection plane C, to thereby enable smooth vertical downward insertion into the collagen band 112 and progressive separation of the collagen band 112 tissue 112, 114 with respect to edge dissection planes E normal to the dissection plane C as the tool 200 is compelled progressively downward into the tissue 112, 114, thereby enabling the provision of clean vertical strip terminal edge walls 504 normal to the clean vertical strip edge wall 502, as illustrated in FIG. 5.

In another aspect, the prongs 228 are staggered and distributed over an overall incision tissue engagement length 280. In the present example wherein the prongs 228 have a maximum width 264 of about 1.6 mm, the overall linear tissue engagement length 280 is about 1.6 cm. Pairs of adjacent prongs 228a, 228b and 228c, 228d are separated from each other by a minimal separation distance 282 in order to ensure that the tool 200 may open and close without the adjacent prongs 228a, 228b and 228c, 228d engaging each other and thereby interfering the operation of the tool; in the present example, the separation distance 282 is about 1 mm. There is also preferably a central prong gap 284 between the prong pairs 228a, 228b and 228c, 228d; in the present example, the prong gap 284 is about 6 mm. Alternative embodiments (not shown) may utilize an equal prong distribution array, wherein the prong gaps 282, 284 have generally equivalent values. What is important is that tool 200 is configured to separate collagen band 112 tissue within the prong gaps 282, 284 along the dissection plane C, through the downward insertion of the prongs 228 into the collagen band 112, and/or through a subsequent spreading movement by the prongs 228.

More particularly, in another aspect, the tool 200 is configured to spread apart and outward the lower workpiece arms 204, 224 and their associated prongs 228 in response to squeezing the upper handle elements 202, 222. Accordingly, some collagen band 112 tissue in the gaps 282, 284 and immediately adjacent to the prongs 228 is initially spread apart and split relative to the dissection plane C in response to downward insertion of the prongs 228 into the collagen band 112 through connection to and movement in correlation with skin 114 and collagen band 112 tissues split by direct engagement with the prongs 228. Next, movement of the prongs 228 outward along directional vectors A1 and A2 in response to squeezing the upper handle elements 202, 222 toward each other along directional vectors H11 and H2 causes the prong outer surfaces 272 to push directly-engaged skin 114 and collagen band 112 tissue outward. Adjacent gelatinous collagen band 112 tissues within the gaps 282, 284 connected to the pushed tissues are also thus urged to move outward, causing further splitting of the collagen band 112 relative to the dissection plane C, the remaining un-split collagen band 112 tissue within the prong gaps 282, 284 thus separating along the dissection plane C.

In another aspect, the preferred curvilinear profile of the prong outer surfaces 272 is configured to further aid in collagen band 112 tissue separation along the dissection plane C; by curving outward, the prong outer surface 272 areas proximal to the prong tips 260 are compelled to move farther away from the dissection plane C as the lower workpiece arms 204, 224 spread than those more distal. In the present embodiment, the prong outer surface bottom halves 227 are generally aligned parallel orientation O spaced 291 from the dissection plane C when the prongs 228 are spread to the maximum outward spacing 290, thus urging the collagen band 112 to be split into two parallel vertical wall portions aligned with the prong outer surfaces 272.

Accordingly, one insertion and spreading evolution with the present tool 200 may produce a neat, vertical dissection of 1.6 cm of donor strip length, which distributes the tissue spreading forces over a larger area than blunt hemostat-type of tools, thereby minimizing blunt tissue trauma. Although any number of the sharpened prongs 228 and lesser or greater overall prong span dimensions 280 may be practiced according to the present invention, the present example illustrates a preferred embodiment for a hand-held and operated tool 200. While more or larger prongs 228 and/or longer linear tissue engagement lengths 280 may enable separation of greater amounts of tissue for each evolution, engaging and splitting more tissue requires correspondingly more force by the surgeon's hand, which may add to the surgeon's fatigue or otherwise make use of the tool inefficient and/or unwieldy. However, it is to be understood that alternative embodiments of the present invention may successfully incorporate greater pluralities of prongs, larger prongs profiles and/or longer linear tissue engagement lengths, particularly if mechanical power-assist means are incorporated (not shown). Accordingly, it is to be understood that the tool 200 described so far illustrates only one embodiment of the present invention, and alternative embodiments will be readily apparent to one skilled in the art.

It will be readily apparent that the tool 200 is much more ergonomically efficient than a conventional hemostat-type of tool, wherein a surgeon's hand must instead spread outward the hemostat handles in order to spread the lower hemostat jaws outward, a difficult and uncomfortable procedure in generating the forces necessary to spread apart the upper skin 114 and collagen band 112 tissues. Instead, the present tool 200 utilizes the much stronger muscles of the surgeon's forearm to easily and progressively squeeze the upper handle elements 202, 222 together and thereby cause the prongs 228 to be leveraged outward and thereby separate the donor strip and remaining collagen band 112 tissues. Moreover, as is really apparent by locating the pivot point 130 more proximal to the prongs 228, forces applied to the upper handle elements 202, 222 are translated and amplified to the prongs 228 through well-known principles of mechanical leverage. In another aspect, locating the pivot point 130 more proximal to the prongs 228 provides that distances traveled in movement of the upper handle elements 202, 222 toward or away from each other are proportionally reduced (and associated forces relative to distance traveled proportionally increased) when translated to the prongs 228, again through well-known mechanical leverage principles. In one aspect, this further provides for a reduction in tissue damage through inadvertent or excessive hand motion, wherein a margin of error is provided for the surgeon such that travel distance of the upper handle elements 202, 222 is translated to the corresponding reduced translated motion of the prongs 228. In another aspect, through leverage, the forces applied by the surgeon's hand to the upper handle elements 202, 222 are proportionally multiplied when translated to outward motion of the prongs 228, thereby providing efficient amplification of the surgeon's muscle forces in direct application to tissue separation, the amplification increased proportionately as the pivot point is located more proximate the prongs 228 than the upper handles 202, 222.

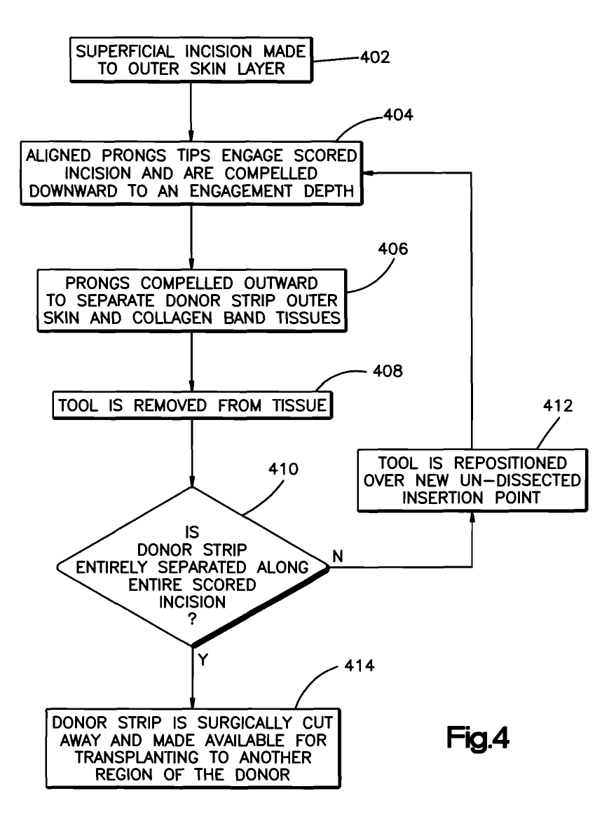

FIG. 4 provides a flowchart illustration of one exemplary method for donor strip harvesting using the tool 200 according to present invention. At 402, a superficial incision made to the outer skin layer tissue 114, preferably to a depth 124 through the entire thickness 122 of the upper skin layer 114 and into an upper region of the collagen band 112. In one example, the superficial incision depth 124 is from about 1.5 mm to about 2 mm, well above the follicle bulbs 102. Thus, in one aspect by keeping the depth of the initial incision above critical areas within the follicular units, follicular transection by the initial incision is prevented, in contrast to conventional strip harvesting techniques that use deep incisions of about 5 mm or more with bladed instruments, which necessarily result in the transection of follicular units directly engaged by the bladed instrument.

In one aspect, the superficial incision at 402 may be performed with a double bladed instrument with a depth controller; in one example, a conventional double-bladed scoring instrument is modified with a depth controlling means to limit the depth of the scoring to about 1.5 mm to about 2 mm, wherein a conventional depth-limiting means enables minimum incision depths of about 5 mm or more. In another aspect, since the depth of the incision is limited, the superficial incision may be made relatively quickly, in contrast to prior methods using deeper incisions which necessarily require greater care and a more deliberate speed in order to avoid follicular transection. Moreover, where a double-bladed instrument is used, the time required to make the pair of superficial incisions defining the length of the donor strip to be harvested is further cut in half. Uniformity of donor strip width along the length of the strip incision is also accomplished.

Next, at 404, the tool 200 prong tips 260 aligned to form the linear puncture point edge 250 engage the scored incision and are compelled downward to puncture into the skin tissue to a depth about equal to the projection length 262 of the prongs 228 below the lower workpiece arms 204, 224, the prong outer surfaces 270 thereby progressively and gradually separating the tissues 114 and 112 with increasing depth of insertion as described above. In one aspect, the projection length 262 correlates to a depth of insertion 126 extending through the collagen band 112 and encompassing the entire follicular unit 100.

Once fully inserted into the dermal tissue, the staggered prongs 228 are compelled outward at 406 by squeezing the handles 202, 222 together, preferably to their maximum spacing 290, thereby achieving corresponding separation of the outer skin 114 and collagen band 112 tissues along the entire length of the overall linear tissue engagement length 280, as described above. What is important is that as the gelatinous collagen band 112 tissue separates the hair follicles 102, sebaceous glands 104 and other follicular unit elements are compelled to cleanly separate into either the donor strip collagen band or the remaining dermal tissue collagen band without mechanical transection, and thereby the follicular unit elements may be cleanly harvested in a resultant donor strip with a minimum of transection or other soft tissue damage.

At 408, the surgeon relaxes his hand and allows the upper handle elements 202, 222 to be compelled outward again by the resilient spring members 240, 242, thereby causing the prongs 228 to be brought back into the closed linear alignment 250, and the tool 200 is removed from the dermal tissue from its present insertion point. At 410, if the entire donor strip is not yet dissected along the entire incision length, then at 412 the tool 200 is inserted into a new insertion point along the incision to separate an additional donor strip segment, and steps 404-408 are repeated.

When the entire donor strip as defined by the step 402 incision is separated along the vertical plane(s) normal to the superficial incisions according to the present invention, at 414 the donor strip is surgically cut away at its bottom layer interface to the donor tissue region and made available for transplanting to another region of the scalp or other transplantation destination, as will be readily understood by one skilled in the art.

What is important is that the present invention enables the clean separation and division of hair follicle bulbs along the donor strip and the remaining tissue, with a minimal tearing or damaging of individual hair follicle bulbs. FIG. 5 provides a side view of a donor strip 500 obtained with the articles and methods of the present invention. As is readily apparent, the strip edge wall 502 collagen band 112 evidences a plurality of intact and undamaged hair follicles 102 and associated sebaceous glands 114.

In another aspect, the present invention provides advantages for donor strip acquisition involving more difficult tissue types. More particularly, it is known that dense and curly hair follicle arrays, for example those presented by African-American and some Middle Eastern donor subjects, are difficult to separate through conventional methods without damaging the tightly interwoven hair follicle bulbs. The techniques and articles described above may readily separate these conventionally difficult-to-separate follicular units.

In another aspect, the present invention provides advantages in dealing with hard-to-see hair follicle tissues, such as those involving white or gray-haired donor tissues. Since the articles and methods according to the present invention use superficial shallow incisions above the more deeply-occurring hair follicles, there is no requirement that the hair follicle bulbs or other tissue elements be visually identified during incision steps in order to avoid damage thereto, as is generally required in conventional methods.

Detailed descriptions of the preferred embodiment are provided herein. It is to be understood, however, that the present invention may be embodied in various forms. For example, although the embodiments have been described with respect to donor strip harvesting for techniques related to male pattern baldness issues, dermal strips may be acquired according to the present invention for other purposes. In another aspect, the present invention is also applicable to other types of surgical procedures. In one example, the invention may be used for making a scalp incision for a face lift; in another example, the invention may be used for making an excision; other applications will be apparent to one skilled in the art. Therefore, specific details disclosed herein are not to be interpreted as limiting, but rather as a basis for the claims and as a representative basis for teaching one skilled in the art to employ the present invention in virtually any appropriately detailed system, structure or manner.

Other objects and advantages of the present invention will become apparent from the following descriptions, taken in connection with the accompanying drawings, wherein, by way of illustration and example, an embodiment of the present invention is disclosed.

What is claimed is:

1. A tool, comprising:
a first handle element having a first upper handle, a first lower workpiece arm, and a first at least one prong extending downward from the first lower workpiece arm, each of the at least one prongs having a bottom sharpened point;
a pivot pin rotatably connected to the first handle element between the first upper handle and the first lower workpiece arm;
a second handle element having a second upper handle, a second lower workpiece arm and a second at least one prong extending downward from the second lower workpiece arm, each of the at least one prongs having a bottom sharpened point, the second handle element rotatably connected to the pivot pin between the second upper handle and the second lower workpiece arm;
wherein the first and second upper handle elements are configured to pivot the first workpiece arm toward or away from the second workpiece arm in response to an opposite motion of the first upper handle toward or away from the second upper handle outward;
wherein the first and second at least one prongs are configured to align the sharpened prong points into a linear spaced edge array when the first and second lower workpiece arms are brought together in a closed position, the prongs further aligned vertically over the linear spaced edge array and generally along a center dissection plane; and
wherein the prongs each comprise an outer curvilinear surface configured to engage workpiece tissue and move the engaged tissue outward away from the center dissection plane as the first lower workpiece arm is compelled away from the second lower workpiece arm.

2. The tool of claim 1 wherein at least one of the first and second at least one prongs is a plurality of spaced apart prongs.

3. The tool of claim 2 wherein the first at least one prong is a first plurality of spaced apart prongs, and the second at least one prong is a second plurality of spaced apart prongs.

4. The tool of claim 3, wherein bottom portions of each of the first plurality prong outer curvilinear surfaces are configured to align in a first prong outer surface planar array generally parallel to and spaced from the dissection plane when the first and second workpiece arms are moved away from each other into an open position; and
wherein bottom portions of each of the second plurality prong outer curvilinear surfaces are configured to align in a second prong outer surface planar array generally parallel to and spaced from the dissection plane and the first prong outer surface planar array when the first and second workpiece arms are moved away from each other into the open position.

5. The tool of claim 4, wherein each of the prongs taper from a widest top region to the sharpened point.

6. The tool of claim 5 wherein:
the prongs each have a downward vertical projection length of from about 5 mm to about 6 mm, a widest top region width dimensions of about 2 mm, and a widest top region thickness dimensions of about 2 mm; and
the vertically aligned first and second pluralities of prongs defining a dissection strip length of about 1.6 cm.

7. The tool of claim 6, wherein the prongs comprise generally planar lateral edge surfaces aligned vertically with respect to the dissection plane.

8. The tool of claim 5, further comprising a mechanical power means connected to the first upper handle and the second upper handle, the mechanical power means configured to pivot the first upper handle relative to the second upper handle about the pivot pin.

9. The tool of claim 5, wherein the first and second upper handles are configured for engagement by a user's hand.

10. The tool of claim 9, further comprising a resilient spring means connected to the first and second handle elements, the spring means configured to urge the first upper handle element to pivot about the pivot pin connection relative to the second upper handle element, the first upper handle thereby urged to pivot away from the second upper handle and the first lower workpiece arm to pivot toward the second lower workpiece arm.

11. A tool for tissue incision, comprising:
an upper handle element;
a pair of spaced parallel blades projecting downward from the upper handle element; and
a depth controller means disposed adjacent to the spaced parallel blades and configured to limit a depth of tissue incision of the spaced parallel blades to from about 1.5 mm to about 2 mm.

12. A method for tissue dissection, comprising the steps of:
making a superficial incision into the tissue;
aligning first and second pluralities of sharpened prongs vertically generally in a dissection plane above the superficial incision, the prongs having wide upper regions tapering to bottom sharpened points, wherein the sharpened points form a spaced linear puncture point edge;
engaging the scored incision with the spaced linear puncture point edge;
moving the tapered prongs downward vertically through the superficial incision into the tissue, prong outer surfaces and the sharpened points thereby progressively and gradually dissecting the tissue along the dissection plane with increasing depth of insertion; and
moving the first prong plurality horizontally outward relative to the second prong plurality, thereby separating the tissue into a first vertical wall tissue portion engaged by first prong plurality outer surfaces and a second vertical wall tissue portion engaged by second prong plurality outer surfaces, wherein the first vertical wall tissue portion is aligned generally parallel to and spaced from the dissection plane and the second vertical wall tissue portion is aligned generally parallel to and spaced from the dissection plane and the first vertical wall tissue portion.

13. The method of claim 12, wherein:
the tissue is skin tissue comprising an upper skin layer and a lower collagen band comprising follicular unit elements; and
the step of making a superficial incision into the tissue comprises cutting thorough the upper skin layer and into an upper area of the lower collagen band above the follicular unit elements.

14. The method of claim 13 wherein the step of making a superficial incision into the tissue comprises the step of limiting the incision depth to from about 1.5 mm to about 2 mm.

15. The method of claim 14 wherein the step of making a superficial incision into the tissue comprises using a double bladed instrument with a depth controller means.

16. The method of claim 13 wherein the step of separating the tissue into first and second vertical wall tissue portions further comprising the steps of:
compelling a first plurality of follicular unit elements to separate into the first vertical wall tissue portion without transection; and
compelling a second plurality of follicular unit elements to separate into the second vertical wall tissue portion without transection.

17. The method of claim 16, further comprising the steps of:
compelling tissue located at first prong plurality gaps between adjacent spaced prongs to separate into the first vertical wall tissue portion, thereby compelling a first plurality of gap tissue follicular unit elements to separate into the first vertical wall tissue portion without transection; and
compelling tissue located at second prong plurality gaps between adjacent spaced prongs to separate into the second vertical wall tissue portion, thereby compelling a second plurality of gap tissue follicular unit elements to separate into the second vertical wall tissue portion without transection.

18. The method of claim 17, further comprising the steps of:
extending the first plurality of prongs downward from a first handle element lower workpiece arm, the first handle element having a first upper handle;
rotatably connecting a pivot pin to the first handle element between the first upper handle and the first lower workpiece arm;
extending the second plurality of prongs downward from a second handle element lower workpiece arm, the second handle element having a second upper handle;
rotatably connecting the second handle element to the pivot pin between the second upper handle and the second lower workpiece arm; and
wherein the step of moving the first prong plurality horizontally outward relative to the second prong plurality comprises the steps of:
compelling the upper first and second handles toward each other;
the first workpiece arm pivoting away from the second workpiece arm about the pivot pin in response to the step of compelling the upper first and second handles toward each other.

19. The method of claim 18, further comprising the steps of:
configuring the first and second upper handles for engagement by a user's hand;
locating the pivot pin more proximate to the first and second prong pluralities than the first and second upper handles;
wherein the step of compelling the upper first and second handle elements toward each other comprises the steps of:
engaging the first upper handle with a thumb;
engaging the second upper handle with fingers opposing the thumb; and
squeezing the thumb and opposing fingers together with a squeezing force; and
the pivot pin leveraging the squeezing force into an outward prong spreading force expended by the prong outer surfaces against the tissue, the outward prong spreading force greater than the squeezing force.
